(19) United States
(12) Patent Application Publication
(10) Pub. No.: US 2007/0161057 A1
(43) Pub. Date: Jul. 12, 2007

(54) Methods, compositions, and kits for detecting cyrptosporidium pathogens

(76) Inventors: Eddie P. Jeffries III, Sterling, VA (US); Charles Clifford Young, Mount Airy, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/572,054

(22) PCT Filed: Sep. 8, 2004
(86) PCT No.: PCT/US04/29087

§ 371 Date: Dec. 6, 2006

Related U.S. Application Data
(60) Provisional application No. 60/503,362, filed on Sep. 16, 2003.

(51) Int. Cl. G01N 33/569

(52) U.S. Cl. 435/7.22

(57) ABSTRACT

The invention provides an efficient, sensitive, and reliable method for detection of parasites such as Cryptosporidium by efficiently extracting molecular markers or antigens using non-ionic detergents and ECL detection, and kits and compositions for performing such methods.

### I. BACKGROUND

This application claims the benefit of priority to U.S. Provisional Patent Application No. 60/503,362, filed Sep. 16, 2003, which is incorporated herein by reference in its entirety.

Cryptosporidium is a genus of food and water-borne parasites that infect humans and animals causing severe intestinal distress (C. Drozd et al. (1996) Appld. Eviron. Micro., 62(4): 1227-1232). The infection is transmitted in the form of an oocyst, primarily via fecal-oral contact. While, generally, not life threatening to humans, cryptosporidium infection can be extremely dangerous to the immunocompromised. Recent outbreaks of Cryptosporidium parvum (C. parvum) infections due to contaminated water have emphasized the importance of detecting cryptosporidium in water, food, environmental samples, humans, animals, and other potential sources of contamination.

Methods for the detection of Cryptosporidium oocysts are described in U.S. Pat. No. 6,146,838 to Williams et al. and in U.S. Pat. No. 6,475,747 to Tsang et al., which are incorporated herein by reference. Both patents describe solubilizing antigens from Cryptosporidium oocytes and detecting the solubilized antigens. U.S. Pat. No. 6,146,838 specifically describes solubilization in the presence of bile salts and/or ionic detergents. U.S. Pat. No. 6,475,747 describes oocyst solubilization using zwitterionic detergents. Both patents also describe using an electrochemiluminescence (ECL) immunoassay for detecting the solubilized antigens.

Improved methods for the detection of Cryptosporidium oocysts are presented herein.

### II. SUMMARY OF THE INVENTION

The present invention provides an efficient, sensitive, and reliable method for the detection of Cryptosporidium parasites.

One embodiment of the invention is a method for measuring Cryptosporidium , e.g., Cryptosporidium oocysts and/or sporozoites, in a sample where the sample is incubated in an extraction medium for a period of time sufficient to extract, and optionally solubilize, into the extraction medium, a marker or markers of Cryptosporidium , e.g., a carbohydrate, protein, or glycoprotein marker.

The extraction medium contains an extraction reagent comprising a non-ionic alkyl-polyoxyethylene detergent of general formula R—(OCH₂CH₂)ₙ—O—Z, where (i) R is —H or —CH₃; (ii) n is an integer greater than 2; and (iii) Z is an alkyl group, for example, —(CH₂)ₘCH₃, where m is between 7 and 17. In certain embodiments of the invention, the extraction medium contains an extraction reagent comprising a non-ionic alkyl-polyoxyethylene detergent of general formula R—(OCH₂CH₂)ₙ—O—Z, where (i) R is —H; (ii) n is an integer between 8 and 23; and (iii) Z is —(CH₂)ₘCH₃, where m is between 7 and 17. In various embodiments of the invention, the extraction medium contains an extraction reagent comprising a non-ionic alkyl-polyoxyethylene detergent of general formula R—(OCH₂CH₂)ₙ—O—Z, where (i) R is —H; (ii) n is 12; and (iii) Z is —(CH₂)ₘCH₃, where m is 11. In certain embodiments, extraction reagents may comprise H—(OCH₂CH₂)₁₂—O—(CH₂)₁₁CH₃ also known as Laureth-12.

During the extraction of the sample, the concentration of the detergent can be between 0.001-1.0% by weight. In some embodiments, the concentration of detergent can be between 0.002-0.1% by weight. In certain embodiments, the concentration of detergent can be between 0.01-0.05% by weight. Additionally, the extraction medium can comprise a pH buffer. The sample can be incubated in the presence of the extraction medium at temperatures greater than 50° C., such as temperatures greater than 75° C. or 95° C. In some embodiments, the extraction medium temperature may be 100° C. or more.

The extracted Cryptosporidium markers are measured for the purpose of detecting Cryptosporidium in the sample. The measurement of a marker can be performed using an immunoassay, for example, an ECL immunoassay. In certain embodiments, this measurement can comprise forming an assay mixture comprising the extracted, and (optionally) solubilized, marker(s) and an antibody that recognizes the marker(s), incubating this mixture under conditions sufficient to permit binding of the marker(s) to the antibody to form an antibody-antigen complex, and determining the presence of the antigen-antibody complex, thereby measuring Cryptosporidium .

According to certain embodiments of the invention, the product of the extraction step can be buffer-exchanged, such as with a buffered solution optimized for the detection step, prior to carrying out the step for detecting Cryptosporidium . In addition, centrifugation and/or filtration can be used to remove particulate debris from the product of the extraction step prior to detection of the solubilized marker(s). Furthermore, the sample can be concentrated, for example, by filtration or centrifugation, prior to contacting the sample with the extraction reagent.

The invention further relates to kits (and reagent compositions) for measuring Cryptosporidium , e.g., C. parvum, in a sample. According to some embodiments, the kits include, in one or more containers: i) an extraction reagent comprising a non-ionic detergent, e.g., Laureth-12, suitable for use in solubilizing a marker of Crytposporidium oocytes, e.g., oocytes of C. parvum, according to the extraction methods of the invention, and ii) an antibody that binds the marker. An antibody of the present invention can be labeled with a detectable label, for example, an electrochemiluminescent label. The kit can also include a second antibody that binds the marker. The kit can further include a solid phase to which the second antibody is immobilized or capable of being immobilized. In some embodiments, the solid phase is a particle, for example, a magnetizable particle. In another embodiment, the solid phase can be the surface of a slide or a container, for example, the surface of a well in a multi-well plate. In certain embodiments, the solid phase can be the surface of an electrode, for example, a carbon electrode.

### III. DETAILED DESCRIPTION OF THE INVENTION

One aspect of the invention relates to methods for extracting microorganism markers from a sample. Markers refer herein to components of a microorganism that can be measured to determine the quantity or presence of the microorganism in the sample. Extraction refers herein to the processing of a sample to make a marker more accessible for measurement. Measured, as used herein, is understood to encompass quantitative and qualitative measurement, and encompasses measurements carried out for a variety of purposes including, but not limited to, detecting the presence of an analyte, measuring the amount of an analyte, and/or identifying an analyte in a sample. The methods are amenable for use in rapid, easy to carry out methods for measuring microorganisms, e.g., Cryptosporidium , including C. parvum and oocytes of these organisms, in a variety of different types of samples. The extraction methods of the present invention can be used with a wide variety of markers, including proteinacious, carbohydrate, nucleic acid, and/or lipid markers, from a wide variety of microorganisms. In certain embodiments, the markers are antigenic markers, e.g., protein, carbohydrate, and/or glycoprotein-containing antigens present in a pathogenic organism, for example, a protozoa, such as an oocyte-forming protozoa, including members of the Cryptosporidium genus, like C. parvum.

According to some embodiments of the present invention, a sample suspected of being contaminated with a pathogenic organism (a protozoa, such as an oocyte-forming protozoa, e.g., a member of the Cryptosporidium genus like C. parvum) is treated with an extraction reagent that extracts, and optionally solubilizes, a marker from the organism. It is beneficial for the present invention if the marker is unique for the genus or species of interest. The extracted and, optionally solubilized, marker is then measured. Measurement of the extracted marker can allow for detection of the presence, amount and/or identity of the organism in the sample. By way of example, extraction can include the liberation of markers from cells, microorganisms, or organelles, e.g., by (i) rupturing or solubilizing membranes, cell walls, envelopes, etc. to release markers comprised or encased within, attached to, and/or incorporated into the membranes, cell walls, or envelopes, etc., (ii) cleaving a marker from a larger chemical moiety, (iii) breaking down and/or dissolving a polysaccharide coat, and/or (iv) breaking down and/or dissolving a jelly coat. The extraction step can also include liberation of markers, cells, organelles, and/or microorganisms from components of the surrounding sample matrix. The matrix can include the medium in which the organism or the marker is present. In various embodiments, extraction of the sample leads to solubilization of the marker of interest.

According to certain embodiments of the present invention, a sample suspected of being contaminated with Cryptosporidium oocytes is treated with an extraction reagent that solubilizes a marker from Cryptosporidium oocytes in the sample. The marker can be unique to Cryptosporidium , e.g., C. parvum. The solubilized marker can then be measured (during the measurement step). Measurement of solubilized markers allows for the detection of the presence or amount of Cryptosporidium in the sample. In certain embodiments, the measurement results can be specific for the type of marker. By way of example, the measurement of solubilized markers can be specific for Cryptosporidium oocytes, e.g., C. parvum oocytes. Of course, it is understood that even in highly specific assays there may be some number of closely related cross-reacting materials.

In some embodiments, the extraction reagent for use in extraction/solubilization steps of the invention can be a extraction reagent comprising a non-ionic alkyl-polyoxyethylene detergent of general formula R—(OCH₂CH₂)ₙ—O—Z, where i) R is —H or —CH₃; ii) n is an integer greater than 2, such as 8, 12, and 23; iii) Z is an alkyl group, for example —(CH₂)ₘCH₃, where m is between 7 and 17, and in some embodiments 11. Reagents of the present invention can comprise H—(OCH₂CH₂)₁₂—O—(CH₂)₁₁CH₃, also known as Laureth-12 according to the International Nomenclature of Cosmetic Ingredients (INCI).

Examples of detergents useful in the present invention are:
BRIJ 35, 30% Aqueous Solution
BRIJ® 35, PROTEIN GRADE Detergent, 10% Solution, Sterile-Filtered
GENAPOL C-100, PROTEIN GRADE Detergent, 10% Solution, Sterile-Filtered
GENAPOL X-080, PROTEIN GRADE Detergent, 10% Solution, Sterile-Filtered
GENAPOL X-100, PROTEIN GRADE Detergent, 10% Solution, Sterile-Filtered
Octylphenoxypolyethoxyethanol
Octylphenoxypolyethoxyethanol, Hydrogenated
Octylphenoxypolyethoxyethanol, Hydrogenated
Polyethylene Glycol Dodecyl Ether
Polyethylene Glycol Lauryl Ether
Polyoxyethylene (10) Dodecyl Ether
Polyoxyethylene (10) Isotridecyl Ether
Polyoxyethylene (8) Dodecyl Ether
Polyoxyethylene (23) Lauryl Ether
Polyoxyethylene (23) Lauryl Ether
Polyoxyethylene(20) Sorbitan Monolaurate
Polyoxyethleneglycol Dodecyl Ether
Polyoxyethyleneglycol Dodecyl Ether
Polyoxypropylenepolyoxyethylene Block Copolymer

These non-ionic detergents, for example, Laureth-12, show an improved performance in methods for extracting and measuring markers of Cryptosporidium compared with the zwitterionic synthetic compounds and ionic detergents used in the prior art. The reagent of the present invention both produces more markers that are useful and available for analysis and can be used at lower concentrations. The extraction reagent can be primarily aqueous and can also comprise salts and/or pH buffers. In some embodiments, the reagent can be buffered at a pH that is compatible with the formation of antigen-antibody complexes in immunoassays. For example, the pH can be between 6.5 and 8.5. In some embodiments, the pH can be between 7.0 and 8.0. Alternatively, the sample can be buffer-exchanged prior to the detection step, e.g., through dialysis, ultrafiltration, gel filtration, and other methods well known in the art.

In some embodiments of the invention, a sample suspected of containing Cryptosporidium oocysts can be contacted with a buffered solution containing a non-ionic alkyl-polyoxyethylene detergent as described above, e.g., Laureth-12, for a period of time sufficient to solubilize microorganism markers, where the contacting is being carried out at an elevated temperature. In certain embodiments, the contacting is carried out at a temperature greater than 75° C., In other embodiments, the contacting is carried out at a temperature greater than 95° C. or 100° C., followed by detection of the Cryptosporidium marker in the solubilized material using Cryptosporidium test reagents.

The extraction methods of the invention can include concentrating a sample suspected of containing Cryptosporidium oocysts, for example, by filtration or centrifugation, prior to contacting the sample with an extraction reagent and/or prior to the detection step. Also, centrifugation or filtration can be used to remove particulate debris after solubilization but before detection of solubilized markers.

Measurement of extracted markers can be carried out by any of numerous techniques available in the art of biological assays, including but not limited to, nucleic acid hybridization assays, nucleic acid amplification assays, cell culture-based assays, agglutination tests, immunoassays (or other assay formats based on the use of specific binding partners of the marker of interest), immunochromatographic assays, enzymatic assays, etc. In various embodiments, the detection method can be a binding assay, for example, an immunoassay, and the detection step can be performed by contacting an assay composition with one or more detection molecules capable of specifically binding with the marker(s) of interest. In certain embodiments, the assay uses a sandwich or competitive binding assay format. Examples of sandwich immunoassays performed on test strips are described in U.S. Pat. No. 4,168,146 to Grubb et al. and U.S. Pat. No. 4,366,241 to Tom et al., both of which are incorporated herein by reference. Examples of competitive immunoassay devices suitable for use with the present invention include those disclosed by U.S. Pat. No. 4,235,601 to Deutsch et al., U.S. Pat. No. 4,442,204 to Liotta, and U.S. Pat. No. 5,208,535 to Buechler et al., each of which are incorporated herein by reference. In some embodiments, at least one of the binding reagents employed in such an assay can be immobilized on a solid phase support. In some embodiments of the present invention, the pathogenic organism detection can be further improved by using immunoassays with electrochemiluminescent (ECL)-labeled antibodies.

According to a certain embodiments of the invention, samples can be subjected to the extraction method of the invention followed by measurement of marker(s) using ECL-based assay formats, for example, ECL-based immunoassays. The high sensitivity, broad dynamic range, and selectivity of ECL are useful for medical diagnostics purposes. Commercially available ECL instruments have become widely used for various reasons including their excellent sensitivity, dynamic range, precision, and tolerance of complex sample matrices.

In various embodiments, the marker can be measured in a solid phase sandwich immunoassay employing ECL detection. In a solid phase sandwich immunoassay, two antibodies directed against the marker can be used: i) a capture antibody that can be linked or is capable of being linked (e.g., through the formation of a specific binding pair such as a biotin-streptavidin interaction) to a solid phase, and ii) a detection antibody that can be linked or is capable of being linked (e.g., through the formation of a specific binding pair such as a biotin-streptavidin interaction) to a label such as an ECL label. A sample comprising the solubilized marker can be contacted with the two antibodies and the solid phase so that in the presence of the marker, the two antibodies can bind to the marker to form a “sandwich complex” on the solid phase comprising the label. The label on the solid phase can be measured as a proxy for measurement of the marker in the sample.

Certain commercially available instrumentation uses flow cell-based designs with permanent reusable flow cells. Most binding assays carried out on these types of instruments use magnetically responsive particles as a solid phase support for a solid phase binding assay. Immunocomplexes comprising ECL labels that are bound to the particles can be collected on an electrode in the flow cell with the aid of a magnet. The labels on the collected particles can be induced to emit ECL by application of a voltage to the electrode, and the ECL can be measured as a proxy for measurement of the amount of label. The ECL assay method can also comprise the step of introducing an ECL co-reactant prior to application of the ECL inducing voltage.

Recently, ECL instrumentation has been described that uses reagents immobilized on an electrode used to induce ECL (see, e.g., U.S. Pat. Nos. 6,140,045, 6,066,448, 6,090,545, 6,207,369, and Published PCT Appl. No. W098/12539). In this case, the electrode itself is the solid phase support. Multi-well plates having integrated electrodes suitable for such ECL measurements have also been recently disclosed (see, e.g., copending U.S. Application Nos. 10/185, 274 and 10/185,363 (entitled “Assay Plates, Reader Systems and Methods for Luminescence Test Measurements,” each filed on Jun. 28, 2002, and hereby incorporated by reference). These multi-well plates having integrated electrodes include plates having multiple assay domains within a well, wherein multiple binding reagents can be immobilized. The use of multi-well assay plates allows for the parallel processing and analysis of multiple samples distributed in multiple wells of a plate.

Certain embodiments of the present invention employ ECL labels in immunoassays and labeled reagents that comprise ECL labels, such as ECL-labeled antibodies. At this time, there are a number of commercially available ECL labels for analytical measurements. ECL labels, co-reagents, and detection or measuring methods and systems are described in, e.g, U.S. Pat. Nos. 5,093,268, 5,147,806, 5,324,457, 5,591,581, 5,597,910, 5,641,623, 5,643,713, 5,679,519, 5,705,402, 5,846,485, 5,866,434, 5,786,141, 5,731,147, 6,066,448, 6,136,268, 5,776,672, 5,308,754, 5,240,863, 6,207,369, and 5,589,136 and PCT Appin. Nos. W099/63347, W000/03233, W099/58962, W099132662, W099/14599, W098/12539, W097/3 6931, W098/57 154, and PCT/US02/19788, which are all incorporated herein by reference. Examples of ECL labels useful for the present invention include: i) organometallic compounds where the metal originates from the noble metals of group VIII, including Ru-containing and Os-containing organometallic compounds, such as the tris-bipyridyl-ruthenium (RuBpy) moiety, and ii) luminol and related compounds.

Species that participate with the ECL label in the ECL process are referred to herein as ECL co-reactants. Commonly used ECL co-reactants include tertiary amines (see e.g., U.S. Pat. No. 5,846,485, incorporated herein by reference), oxalate, and persulfate for ECL from RuBpy and hydrogen peroxide for ECL from luminol (see, e.g., U.S. Pat. No. 5,240,863, incorporated herein by reference). Additionally, the light generated by ECL labels can be used as a reporter signal in diagnostic procedures (U.S. Pat. No. 5,238,808, Bard et al., incorporated herein by reference). For instance, an ECL label can be covalently coupled to a binding reagent such as an antibody and the participation of the binding reagent in a binding interaction can be monitored by measuring ECL emitted from the ECL label.

### IV. EXAMPLES

The following examples are illustrative of some of the extractions, plates, kits, and methods falling within the scope of the present invention. They are, of course, not to be considered in any way to limit the invention. Numerous changes and modifications can be made with respect to the invention by one of ordinary skill in the art without undue experimentation.

Materials and Methods:

Test Samples:

Cryptosporidium oocysts were purchased from Pleasant Hill Farms and diluted with phosphate buffered saline (PBS, BioWhittaker) to a desired concentration prior to analysis.

Extraction:

A series of extraction reagents were compared. Each consisted of PBS supplemented with either 0.06% Laureth-12, 0.03% Laureth-12, 2% Zwittergente®,0.5% SDS, or 0.25% SDS. The extraction reagents were mixed with tests samples at a 1:1 ratio to a final concentration of 0.03% Laureth-12, 0.015% Laureth-12, 1% Zwittergent®, 0.25% SDS, or 0.125% SDS and incubated at 100° C. for 60 minutes.

Samples were centrifuged at 13,000 rcf for 10 minutes to remove debris and 500 pi1 aliquots were used for analysis. The SDS-containing samples were also treated with either 10% and 5% BSA to eliminate the effect of the detergent on the assay.

### Example 1.

The efficiency of sample preparation was tested using an ECL-based sandwich immunoassay for Cryptosporidium (PATH/GEN® Cryptosporidium parvum Test, available as a kit from Bioveris Corp.). The assay used a biotin-labeled capture antibody and a detection antibody that was labeled with BV-TAGO® NHS Ester (Bioveris Corp.), a derivative of ruthenium(II)-tds-bipyridine. The sample was combined with the TAG-labeled antibody and biotin-antibody pre-bound to streptavidin-coated magnetically-responsive beads and incubated to allow the formation of sandwich complexes on the beads. The resulting bead suspension was analyzed using an ORIGEN® Analyzer (IGEN International) and ECL detection.

The results presented in Tables 1-3 demonstrate that the non-ionic detergent Laureth-12 provides better extraction efficiency at lower detergent concentrations than the zwitterionic detergent Zwittergent® and the ionic detergent SDS. ECL is the raw electrochemiluminescence signal and S/BG is the signal to background ratio (i.e., the ratio of the signal in the presence and absence of solubilized oocytes).

TABLE 1
Detergent concentrations

| | Laureth-12 | | | | Zwittergent ® | |
|---|---|---|---|---|---|---|
| Oocyst/ | 0.03% | | 0.015% | | 1.0% | |
| ml | ECL | S/BG | ECL | S/BG | ECL | S/BG |
| Negative | 7771 | 1.0 | 8222 | 1.0 | 8282 | 1.0 |
| 1 × 10⁴ | 499414 | 64.3 | 554987 | 67.5 | 239106 | 28.9 |
| 1 × 10³ | 129320 | 16.6 | 134282 | 16.3 | 73041 | 8.8 |
| 1 × 10² | 18421 | 2.4 | 21487 | 2.6 | 14248 | 1.7 |
| 1 × 10¹ | 8135 | 1.0 | 9043 | 1.1 | 7189 | 0.9 |

TABLE 2
Detergent concentrations
SDS

| | |
|---|---|
| Oocyst/ | 0.25% |

| ml | ECL | S/BG |
|---|---|---|
| Negative | 2691 | 1.0 |
| 5 × 10³ | 38637 | 14.4 |
| 5 × 10² | 9136 | 3.4 |
| 50 | 3367 | 1.3 |
| 5 | 2800 | 1.0 |

TABLE 3
Detergent concentrations
SDS

| | |
|---|---|
| Oocyst/ | 0.125% |

| ml | ECL | S/BG |
|---|---|---|
| Negative | 7156 | 1.0 |
| 7.5 × 10³ | 112765 | 15.8 |
| 7.5 × 10² | 33164 | 4.6 |
| 75 | 9181 | 1.3 |
| 7.5 | 7287 | 1.0 |

What we claim is:

1. A method for detecting Cryptosporidium in a sample comprising:
(a) combining said sample with an extraction reagent to form an extraction mixture, said extraction reagent comprising a non-ionic detergent of the formula R—(OCH₂CH₂)ₙ—O—Z, where
i) R is —H or —CH₃
ii) n is an integer greater than 2;
iii) Z is an alkyl group;
(b) incubating said extraction mixture for a period of time sufficient to extract one or more markers of Cryptosporidium oocyst; and
(c) measuring said marker or markers.

2. The method of claim 1, wherein R is H, n is between 8 and 23, and Z is —(CH₂)ₘCH₃, and where m is between 7 and 17.

3. The method of claim 1, wherein the method measures a marker selective for Cryptosporidium parvum.

4. The method of claim 1, wherein said non-ionic detergent is Laureth-12.

5. The method of claim 1, wherein said measurement step comprises:
(a) forming an assay mixture comprising
(i) said marker; and
(ii) an antibody specific to said marker; and
(b) incubating said assay mixture under conditions sufficient to permit binding of said antibody to said marker, thereby forming an antibody-marker complex.

6. The method of claim 1, wherein said measurement is performed by immunoassay.

7. The method of claim 6, wherein said detection is performed by ECL immunoassay.

8. The method of claim 1, wherein said extraction reagent further comprises a pH buffer.

9. The method of claim 1, wherein, prior to said measuring step, the extracted mixture is exchanged into a different buffered solution optimized for said measuring step.

10. The method of claim 4, wherein the concentration of Laureth-12 in the extraction mixture is between 0.002 and 0.1% by weight.

11. The method of claim 4, wherein the concentration of Laureth-12 is between 0.01 and 0.05% by weight.

12. The method of claim 1, wherein said marker is a carbohydrate, protein, and/or glycoprotein marker.

13. The method of claim 1, wherein said extraction mixture is incubated at a temperature greater than 50° C.

14. The method of claim 1, wherein said extraction mixture is incubated at a temperature greater than 75° C.

15. The method of claim 1, wherein said extraction mixture is incubated at a temperature greater than 95° C.

16. The method of claim 1, further comprising concentrating Cryptosporidium in said sample prior to combining said sample with said extraction reagent.

17. The method of claim 1, further comprising removing insoluble debris from said extraction mixture prior to measuring said marker.

18. A method for detecting Cryptosporidium in a sample comprising:
(a) extracting the sample by combining the sample with an extraction reagent to form an extraction mixture, the extraction reagent comprising a non-ionic detergent of the formula R—(OCH₂CH₂)ₙ—O—Z, where
i) R is —H or —CH₃;
ii) n is an integer greater than 2;
iii) Z is an alkyl group;
(b) solubilizing in said extraction mixture, an antigen of Cryptosporidium oocysts and/or sporozoites;
(c) forming an assay mixture comprising,
(i) said solubilized antigen; and
(ii) an antibody specific to said antigen;
(d) incubating said assay mixture under conditions sufficient to permit binding of said antibody to said antigen thereby forming an antibody-antigen complex; and
(e) measuring said antibody-antigen complex, thereby measuring Cryptosporidium in the sample.

19. The method of claim 18, wherein R is H, n is between 8 and 23, and Z is —(CH₂)ₘCH₃, and where m is between 7 and 17.

20. The method of claim 18, wherein said marker is selective for Cryptosporidium parvum.

21. The method of claim 18, wherein said non-ionic detergent is Laureth-12.

22. The method of claim 21, wherein the concentration of said Laureth-12 in said extraction mixture is between 0.002 and 0.1% per weight.

23. The method of claim 21, wherein the concentration of said Laureth-12 in said extraction mixture is between 0.01 and 0.05% per weight.

24. The method of claim 18, wherein said extracting is performed at a temperature greater than 50° C. for a period of time sufficient to solubilize the marker of Cryptosporidium oocyst.

25. The method of claim 18, wherein said extracting is performed at a temperature greater than 75° C. for a period of time sufficient to solubilize the marker of Cryptosporidium oocyst.

26. The method of claim 18, wherein said extracting is performed at a temperature greater than 95° C. for a period of time sufficient to solubilize the marker of Cryptosporidium oocyst.

27. The method of claim 18, further comprising concentrating Cryptosporidium in said sample prior to extracting said sample.

28. The method of claim 18, further comprising removing insoluble debris from said extraction mixture prior to measuring said antibody-antigen complex.

29. An improved method for detecting Cryptosporidium in a sample comprising:
(a) combining said sample with an extraction reagent to form an extraction mixture;
(b) incubating said extraction mixture for a period of time sufficient to solubilize a marker of Cryptosporidium oocysts; and
(c) measuring said marker by immunoassay;
the improvement being the use of an extraction reagent comprising a detergent selected from nonionic detergents of the chemical formula:
R—(OCH₂CH₂)ₙ—O—Z, where
i) R is —H or —CH₃;
ii) n is an integer greater than 2; and
iii) Z is an alkyl group.

30. The method of claim 29, wherein R is H. n is between 8 and 23, and Z is —(CH₂)mCH₃, and where m is between 7 and 17.

31. The method of claim 29, wherein said marker is the selective marker C. panvum.

32. The method of claim 29, wherein said non-ionic detergent is Laureth-12.

33. The method of claim 32, wherein the concentration of said Laureth-12 in said extraction mixture is between 0.002 and 0.1% per weight.

34. The method of claim 32, wherein the concentration of said Laureth-12 in said extraction mixture is between 0.01 and 0.05% per weight.

35. The method of claim 29, wherein said solubilizing is performed at a temperature greater than 50° C. for a period of time sufficient to solubilize a marker of Cryptosporidium oocyst.

36. The method of claim 29, wherein said solubilizing is performed at a temperature greater than 75° C. for a period of time sufficient to solubilize a marker of Cryptosporidium oocyst.

37. The method of claim 29, wherein said solubilizing is performed at a temperature greater than 95° C. for a period of time sufficient to solubilize a marker of Cryptosporidium oocyst.

38. The method of claim 29, further comprising concentrating Cryptosporidium in said sample prior to combining said sample with said extraction reagent.

39. The method of claim 38, wherein said step of concentrating comprises centrifuging said sample.

40. The method of claim 29, further comprising removing insoluble debris from said extraction mixture prior to measuring said marker.

41. The method of claim 40, wherein said step of removing insoluble debris comprises centrifuging said extraction mixture.

42. A kit for measuring Cryptosporidium in a sample comprising in one or more containers:
(a) an extraction reagent comprising a nonionic detergent of general formula R—(OCH₂CH₂)ₙ—O—Z, where
i) R is —H or —CH₃;
ii) n is an integer greater than 2; and
iii) Z is an alkyl group; and
(b) an antibody that binds to a marker of Cryptosporidium oocytes; wherein said extraction reagent is suitable for extracting Cryptosporidium so as to solubilize said marker.

43. The kit of claim 42, wherein said extraction reagent is Laureth-12.

44. The kit of claim 43, further comprising an electrochemiluminescent label.
